(19) United States
(12) Patent Application Publication
(10) Pub. No.: US 2021/0195833 A1
(43) Pub. Date: Jul. 1, 2021

(54) SYSTEM AND METHOD USING NIR SENSING TO CONTROL HEIGHT OF CUTTING ELEMENT ON AGRICULTURAL MACHINE

(71) Applicant: AGCO Corporation, Duluth, GA (US)

(72) Inventor: Kevin J. Hamilton, Hesston, KS (US)

(73) Assignee: AGCO Corporation, Duluth, GA (US)

(21) Appl. No.: 17/137,548

(22) Filed: Dec. 30, 2020

Related U.S. Application Data
(60) Provisional application No. 62/954,782, filed on Dec. 30, 2019.

(51) Int. Cl. A01D 34/00; G01N 21/55; A01D 34/28

(52) U.S. Cl. CPC A01D 34/006; A01D 34/14; A01D 34/283; G01N 21/55

(57) ABSTRACT

A system and method for automatically and dynamically controlling a height of a cutting element on a swather or other agricultural machine based on feedback from NIR testing of the cut plant material. An NIR testing system receives and analyzes NIR reflected by the material cut at a particular cut height, and generates an actual value for an RFV or other property of interest. A user interface allows for entering a desired target value. A cut controller compares the actual value and the target value, and adjust (e.g., raises or lowers) a cutting element (by, e.g., changing a pitch of the cutting element) to change the particular cut height if the actual value for the property is different from the target value. If the actual value is substantially similar to the target value, then the cut controller maintains the position of the cutting element.

### CROSS REFERENCE TO RELATED APPLICATION

This application claims the benefit of U.S. Provisional Application No. 62/954,782, filed Dec. 30, 2019, which is hereby incorporated by reference in its entirety.

### FIELD

The present invention relates to systems and methods for cutting plant material, and more particularly, embodiments concern a system and method for automatically and dynamically controlling a height of a cutting element on a swather or other agricultural machine based on feedback from near infra-red testing of the cut plant material.

### BACKGROUND

A swather (or windrower or mower) is an agricultural machine configured to cut plant material growing in a field and deposit the cut plant material in windrows (or swaths) on the field to dry. An example swather is the Massey Ferguson WR9980 self-propelled mower. Yield, measured in tons per acre, and quality, which may be expressed as a “relative feed value” (RFV), a protein content value, a fiber content value, a “total digestible nutrient” (TDN) value, an “acid detergent fiber” (ADF) value, and a “neutral detergent fiber” (NDF) value, are the two main factors that influence the amount of revenue per acre a forage producer may receive. Often, buyers of premium forage, such as the dairy or export industries, require a minimum RFV score, and if the forage does not meet the premium market it is sold into other uses at a significantly reduced price. The height at which plants are cut during harvesting strongly correlates with quality. In particular, as cut height increases, less tonnage is harvested (because more of the fibrous stem material is left in the field) and the RFV score increases, and as cut height decreases, more tonnage is harvested and the RFV score decreases.

It is known to test samples of the plant material in order to determine properties (e.g., protein content, fiber content, moisture content) that are relevant to its sale or use value. Typically, such testing is done on the plant material after it has been baled. Once a number of bales have been created, a core sample is taken from one of the bales and sent to a third-party laboratory for, e.g., near-infrared (NIR) testing to determine these properties. In an NIR testing system, light having wavelengths between 780 nm and 2500 nm is emitted by the instrument and then reflected by the plant material before being received back into the instrument; filtered, and converted to a voltage or current; and analyzed then to determine the properties of the plant material.

This background discussion is intended to provide information related to the present invention which is not necessarily prior art.

### SUMMARY

Embodiments address the above-identified and other problems and limitations in the prior art by providing a system and method for automatically and dynamically controlling a height of a cutting element on a swather or other agricultural machine based on feedback from NIR testing of the cut plant material, and thereby more closely achieving a target value for one or more properties of the cut plant material.

In one embodiment, a system is provided for automatically and dynamically controlling a height of a cutting element on a swather or other agricultural machine based on feedback from near infra-red sensing of a cut plant material. The system may be incorporated into a swather machine configured to cut a plant material, wherein the swather may include a cutting mechanism having a cutting element configured to cut the plant material at a particular cut height, and the system may include an NIR testing system and a cut controller. The NIR testing system may be configured to emit near-infrared radiation and then receive a reflected response from the plant material cut at the particular cut height, analyze the reflected response, and generate evaluation data including an actual value for a property of the plant material cut at the particular cut height. The cut controller may be operably coupled to the cutting element to control the particular cut height at which the plant material is cut based at least in part on a comparison of the actual value for the property and a target value for the property, including adjusting a height of the cutting element to change the particular cut height if the actual value for the property is substantially different from the target value, and maintaining the particular cut height if the actual value for the property is within a preestablished percentage of the target value.

In yet another embodiment, a method is provided for automatically and dynamically controlling a height of a cutting element on a swather or other agricultural machine based on feedback from near infra-red sensing of a cut plant material. The method may be employed on a swather machine configured to cut a plant material, wherein the swather may include a cutting mechanism having a cutting element configured to cut the plant material at a particular cut height, and the method may include the following steps. An NIR testing system may emit near-infrared radiation and then receive a reflected response from the plant material cut at the particular cut height, analyze the reflected response, and generate evaluation data including an actual value for a property of the plant material cut at the particular cut height. A cut controller may control the particular cut height at which the plant material is cut, including comparing the actual value for the property and a target value for the property and automatically adjusting a height of the cutting element of the swather to change the particular cut height if the actual value for the property is substantially different from the target value, and maintaining the particular cut height if the actual value for the property is within a preestablished percentage of the target value. The system may further include a user interface configured to facilitate a user entering the target value for the property of the plant material.

Various implementations of the above-described embodiments may include any one or more of the following features. The property of the plant material may be an RFV, a protein content value, a fiber content value, a TDN value, an ADF value, and an NDF value. The cut controller may not raise the cutting element above a maximum height, and may not lower the cutting element below a minimum height. The cut controller may adjust the height of the cutting element by actuating a hydraulic component which controls the height of the cutting element. The preestablished percentage may be equal to or less than ten percent of the target value.

This summary is not intended to identify essential features of the present invention, and is not intended to be used to limit the scope of the claims. These and other aspects of the present invention are described below in greater detail.

### DRAWINGS

Embodiments of the present invention are described in detail below with reference to the attached drawing figures, wherein:

FIG. 1 is a cut-away side elevation view of an example swather machine configured to cut plant material growing in a field;

FIG. 2 is a high-level block diagram of an embodiment of a system for automatically and dynamically controlling a height of a cutting element on a swather (of, e.g., FIG. 1) or other agricultural machine based on feedback from near infra-red sensing of the cut plant material;

FIG. 3 is an isometric view of a swathboard component of the swather of FIG. 1, showing an NIR sensor component of the system of FIG. 2; and

FIG. 4 is a flowchart of steps in an embodiment of a method for automatically and dynamically controlling a height of a cutting element on a swather (of, e.g., FIG. 1) or other agricultural machine based on feedback from near infra-red sensing of the cut plant material.

The figures are not intended to limit the present invention to the specific embodiments they depict. The drawings are not necessarily to scale.

### DETAILED DESCRIPTION

The following detailed description of embodiments of the invention references the accompanying figures. The embodiments are intended to describe aspects of the invention in sufficient detail to enable those with ordinary skill in the art to practice the invention. Other embodiments may be utilized and changes may be made without departing from the scope of the claims. The following description is, therefore, not limiting. The scope of the present invention is defined only by the appended claims, along with the full scope of equivalents to which such claims are entitled.

In this description, references to “one embodiment,” “an embodiment,” or “embodiments” mean that the feature or features referred to are included in at least one embodiment of the invention. Separate references to “one embodiment,” “an embodiment,” or “embodiments” in this description do not necessarily refer to the same embodiment and are not mutually exclusive unless so stated. Specifically, a feature, component, action, step, etc. described in one embodiment may also be included in other embodiments, but is not necessarily included. Thus, particular implementations of the present invention can include a variety of combinations and/or integrations of the embodiments described herein.

Embodiments provide a system and method for automatically and dynamically controlling a height of a cutting element on a swather or other agricultural machine based on feedback from NIR testing of the cut plant material, and thereby more closely achieving a target value for one or more properties of the cut plant material. In more detail, an NIR testing system may scan and analyze the plant material being cut and provide an actual value which may then be used as feedback to automatically and dynamically control the cut height of the cutting element within minimum and maximum limits. In one implementation, in which the actual value changes directly with the cut height (e.g., an increase in the cut height results in an increase in the actual value), if the actual value is below a desired target value, a cut controller may increase the height of the cutting element until the actual value reaches either the desired target value or the cutting element reaches its maximum height. If the actual value is above the desired target value, the cut controller may decrease the height of the cutting element until the actual value reaches either the desired target value or the cutting element reaches its minimum height. In another implementation, in which the actual value changes inversely with the cut height (e.g., an increase in the cut height results in a decrease in the actual value), if the actual value is below a desired target value, the cut controller may decrease the height of the cutting element until the actual value reaches either the desired target value or the cutting element reaches its minimum height. If the actual value is above the desired target value, the cut controller may increase the height of the cutting element until the actual value reaches either the desired target value or the cutting element reaches its maximum height.

In one implementation, a user interface may allow the operator to set the desired target value. In one implementation, an NIR sensor component of the NIR testing system may be located after a conditioning roller component and on a swathboard or forming shield component of the swather. In one implementation, the height of the cutting element may be controlled using a header pitch control mechanism, and calibrated using an existing potentiometer or other feedback mechanism. The cut controller may adjust the cut height by actuating a hydraulic third link component of the header pitch control mechanism which controls a pitch (and thereby a height) of the cutting element.

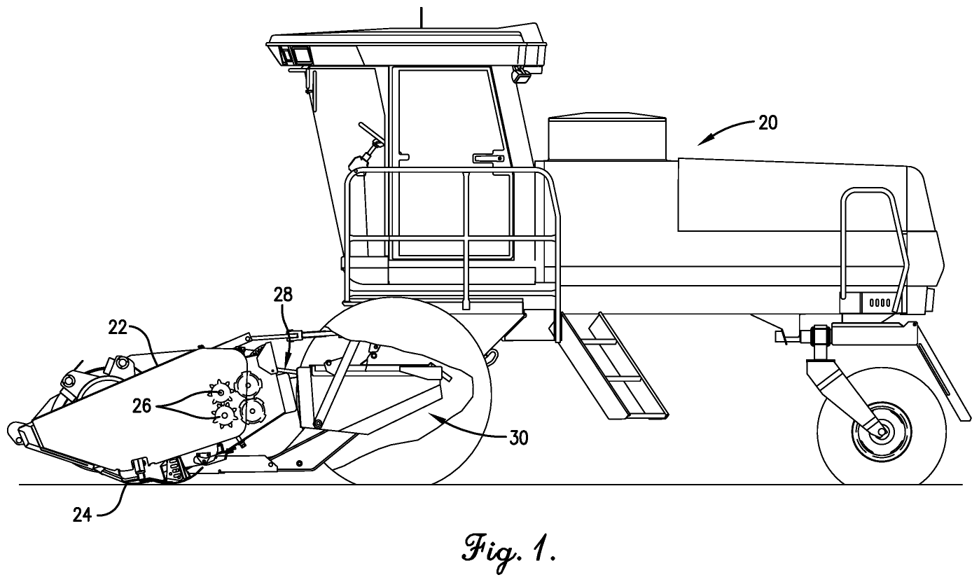

Referring to FIG. 1, an example swather machine 20 is shown into which embodiments of the present invention may be incorporated. Although the example swather 20 is a self-propelled swather, it will be appreciated that embodiments of the present invention may be incorporated into other types of swathers (e.g., towed) with few or no changes. Broadly, the swather 20 may be configured to move over a field, cut plant material, and deposit the cut plant material in windrows on the field. The swather 20 may generally include a header assembly 22, a cutting mechanism 24, conditioning rollers 26, a swathboard 28, and forming shields 30. The header assembly 22 may house the other components of the cutting and windrowing system. The cutting mechanism 24 may be configured to cut the plant material, and may include one or more cutting elements appropriate to the nature of the plant material being cut. The cutting mechanism may be substantially any suitable design, such as a rotary or sickle-type mechanism. The conditioning rollers 26 may be configured to condition (e.g., crush, macerate) the cut plant material, which facilitates drying. The swathboard 28 may be configured to assist in directing the conditioned plant material back to the ground and shaping the windrows (especially to have a generally wider width). The forming shields 30 may be configured to further assist in directing the conditioned plant material back to the ground and shaping the windrows (especially to have a generally narrower width). Thus, the cutting mechanism 24 cuts the plant material at a particular cut height, the conditioning rollers 26 condition the cut plant material, and the swathboard 28 and forming shields 30 direct the conditioned plant material back to the ground and shape the windrows.

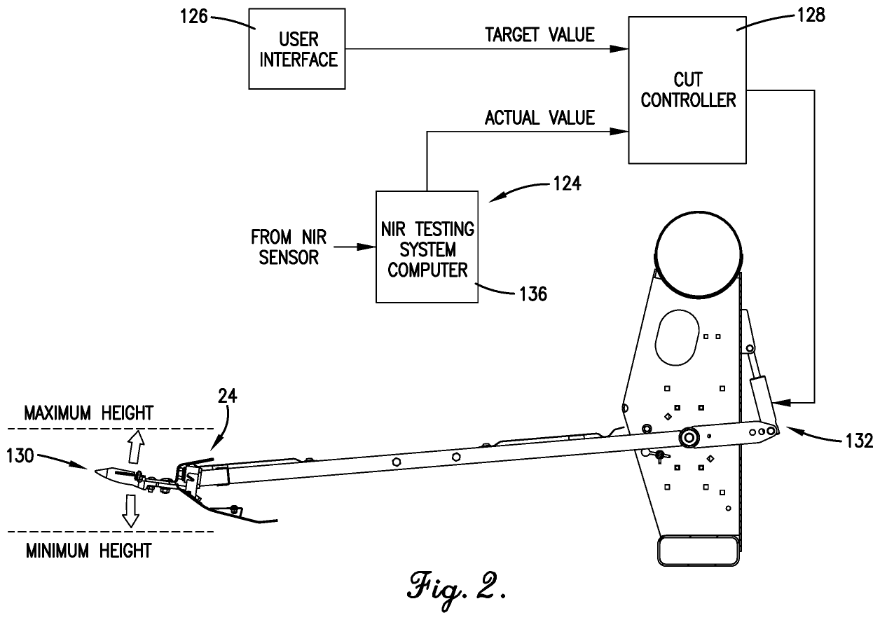

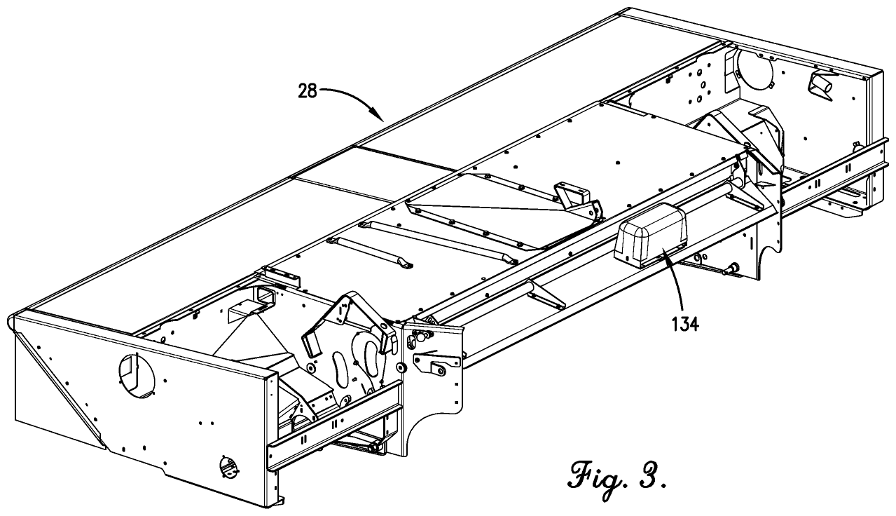

Referring also to FIGS. 2 and 3, an embodiment of a system 120 is shown for automatically and dynamically controlling a height of a cutting element on a swather or other agricultural machine based on feedback from near infra-red testing of the cut plant material. The system 20 is shown incorporated into an example operating environment. The system 120 may comprise some or all of the swather machine 20, including the cutting mechanism 22, an NIR testing system 124, a user interface 126, and a cut controller 128, which may function in accordance with the method 220 described below. As discussed, the swather 20 may be configured to cut plant material growing in a field, and may be otherwise substantially conventional in design, construction, and operation.

As discussed, the cutting mechanism 24 may be configured to cut the plant material, and may include one or more cutting elements 130 appropriate to the nature of the plant material being cut. The cutting mechanism 24 may be substantially any suitable design, such as a rotary or sickle-type mechanism. The cutting mechanism 24 may further include or be associated with a cut height adjustment mechanism 132 configured to adjust a height of at least the cutting element 130 in order to change the height at which the plant material is cut. In one implementation, the cut height adjustment mechanism 132 may include a hydraulic, pneumatic, or similarly actuatable third link component which is actuatable to control the height of the cutting element 130.

The NIR testing system 124 may be configured to emit near-infrared radiation and receive a reflected response from the plant material cut at the particular cut height, analyze the reflected response, and generate evaluation information reflecting one or more properties of the plant material cut at the particular cut height. In one implementation, the NIR testing system 124 may include one or more NIR sensors 134 and a computer 136. As seen in FIG. 3, the NIR sensor 134 may be mounted in or on or otherwise incorporated into the swathboard 28 or other suitable area of the header assembly 22, and may be configured to receive, filter, and convert to a voltage or current the near-infrared radiation reflected by the cut (and, possibly, conditioned) plant material, and transmit the voltage or current to the computer 136. The computer 136 may be located on or remotely from the swather 20, and may be configured to receive the voltage or current transmitted by the NIR sensor 134 and analyze the voltage or current to determine the properties of the plant material and generate the evaluation information reflecting those properties, including an actual value for an RFV, a protein content value, a fiber content value, a TDN value, an ADF value, and an NDF value, or other property of interest.

The user interface 126 may include a numeric or alphanumeric keypad, dial, sliding switch, or other input mechanism configured to facilitate a user communicating to the cut controller 128 a desired target value for the property of the plant material. In one implementation, the user interface may be mounted on or in the swather 20, while in another implementation, the user interface may be provided on smartphone or other mobile communications device.

The cut controller 128 may be operably coupled to the cutting element 130 to control the particular cut height at which the plant material is cut by comparing the actual value for the property as determined by the NIR testing system 124 with the target value received from the user interface 126, and adjusting the height of the cutting element 130 (by, e.g., actuating the cut height adjustment mechanism 132) accordingly. In one implementation, in which the actual value changes directly with the cut height, if the actual value is below a desired target value, the cut controller 128 may increase the height of the cutting element 130 until the actual value reaches either the desired target value or the cutting element 130 reaches its maximum height. If the actual value is above the desired target value, the cut controller 128 may decrease the height of the cutting element 130 until the actual value reaches either the desired target value or the cutting element 130 reaches its minimum height. In another implementation, in which the actual value changes inversely with the cut height, if the actual value is below a desired target value, the cut controller 128 may decrease the height of the cutting element 130 until the actual value reaches either the desired target value or the cutting element 130 reaches its minimum height. If the actual value is above the desired target value, the cut controller 128 may increase the height of the cutting element 130 until the actual value reaches either the desired target value or the cutting element 130 reaches its maximum height. If the actual value for the property is substantially the same as (i.e., within a preestablished percentage of) the target value, then the cut controller 128 may be configured to maintain the particular cut height. As used in this context, “substantially the same” or “substantially different” may mean a difference of between nine and eleven (e.g., ten) percent or more percent or a difference of between four and six (e.g., five) percent.

The system 120 may include additional details discussed elsewhere herein, including those discussed below in describing the operating method 220.

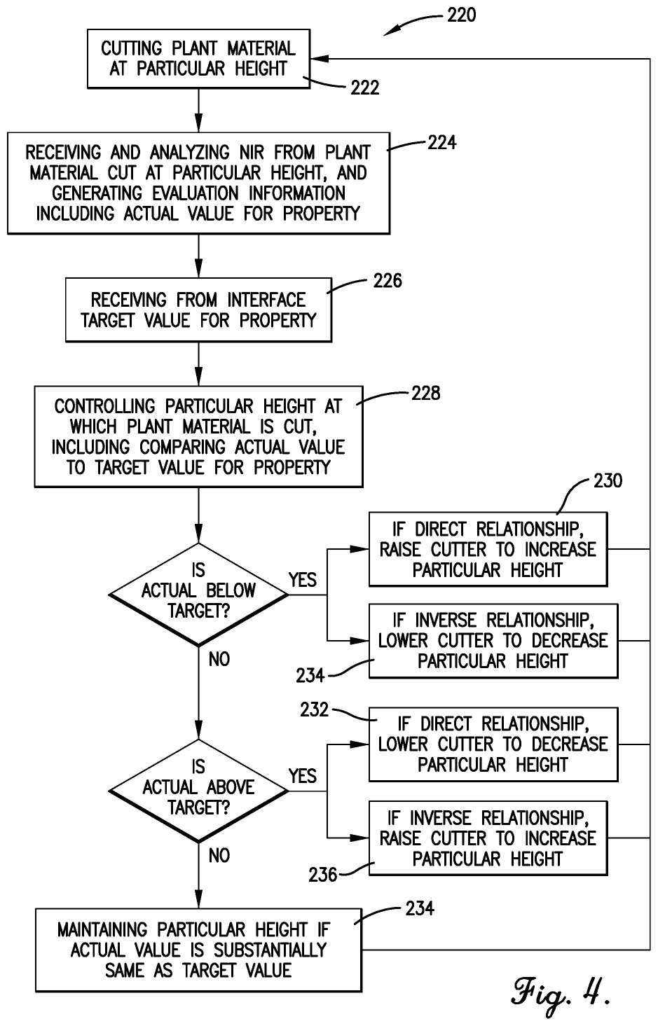

Referring also to FIG. 4, an embodiment of a method 220 is shown for automatically and dynamically controlling a height of a cutting element on a swather or other agricultural machine based on feedback from near infra-red testing of the cut plant material. The method 120 may refer to an example operating environment. The method 220 may comprise some or all of the following steps, which may be implemented by components of the system 120 described above. As discussed, plant material growing in a field may be cut at a particular cut height by a swather machine 20, as shown in step 222. The swather 20 may include a cutting mechanism 24 including one or more cutting elements 130 configured to cut the plant material at the particular cut height.

Near-infrared radiation as emitted by the instrument and as reflected by the plant material cut at the particular cut height may be received and analyzed by an NIR testing system 124, and evaluation data including an actual value for a property of the plant material cut at the particular cut height may be generated by the NIR testing system 124, as shown in step 224. In one implementation, the NIR testing system 124 may include one or more NIR sensors 134 and a computer 136. The NIR sensor 134 may be mounted in or on or otherwise incorporated into a swathboard 28 or other area of a header assembly 22 of the swather 20, and may be configured to receive, filter, and convert to a voltage or current the near-infrared radiation reflected by the cut plant material, and transmit the voltage or current to the computer 136. The computer 136 may be located on or remotely from the swather 20, and may be configured to receive the voltage or current transmitted by the NIR sensor 134 and analyze the voltage or current to determine the properties of the plant material and generate the evaluation information reflecting those properties, including the actual value for a property of interest (e.g., an RFV property).

A desired target value for the property of the plant material may be communicated via a user interface 126 to a cut controller 128, as shown in step 226. The user interface may include a numeric or alphanumeric keypad or other input mechanism configured to facilitate entering the target value. In one implementation, the user interface may be mounted on or in the swather 20, while in another implementation, the user interface may be provided on smartphone or other mobile communications device.

The particular cut height at which the plant material is cut may be controlled by a cut controller 128 by comparing the actual value for the property determined by the NIR testing system 124 with the target value received from the user interface 126 and adjusting the height of the cutting element 130 accordingly, as shown in step 228. In one implementation, in which the actual value changes directly with the cut height, if the actual value is below a desired target value, the cut controller 128 may increase the height of the cutting element 130 until the actual value reaches either the desired target value or the cutting element 130 reaches its maximum height, as shown in step 230. If the actual value is above the desired target value, the cut controller 128 may decrease the height of the cutting element 130 until the actual value reaches either the desired target value or the cutting element 130 reaches its minimum height, as shown in 232. In another implementation, in which the actual value changes inversely with the cut height, if the actual value is below a desired target value, the cut controller 128 may decrease the height of the cutting element 130 until the actual value reaches either the desired target value or the cutting element 130 reaches its minimum height, as shown in step 234. If the actual value is above the desired target value, the cut controller 128 may increase the height of the cutting element 130 until the actual value reaches either the desired target value or the cutting element 130 reaches its maximum height, as shown in step 236.

The method 220 may include additional details discussed elsewhere herein, including those discussed below in describing the implementing system 120.

Although the invention has been described with reference to the one or more embodiments illustrated in the figures, it is understood that equivalents may be employed and substitutions made herein without departing from the scope of the invention as recited in the claims.

Having thus described one or more embodiments of the invention, what is claimed as new and desired to be protected by Letters Patent includes the following:

1. A system incorporated into a swather machine configured to cut a plant material, the swather including a cutting mechanism having a cutting element configured to cut the plant material at a particular cut height, the system comprising:
a near-infrared testing system configured to emit near-infrared radiation and receive a reflected response from the plant material cut at the particular cut height, analyze the reflected response, and generate evaluation data including an actual value for a property of the plant material cut at the particular cut height; and
a cut controller operably coupled to the cutting element to control the particular cut height at which the plant material is cut based at least in part on a comparison of the actual value for the property and a target value for the property, including—
adjusting by the cut controller a height of the cutting element to change the particular cut height if the actual value for the property is substantially different from the target value, and
maintaining by the cut controller the particular cut height if the actual value for the property is within a preestablished percentage of the target value.

2. The system of claim 1, wherein the property of the plant material is selected from the group consisting of: a relative feed value, a protein content value, a fiber content value, a total digestible nutrient value, an acid detergent fiber value, and a neutral detergent fiber value.

3. The system of claim 1, wherein the cut controller adjusts the height of the cutting element by—
raising the cutting element to increase the particular cut height if the actual value for the property is below the target value,
lowering the cutting element to decrease the particular cut height if the actual value for the property is above the target value.

4. The system of claim 1, wherein the cut controller—
does not raise the cutting element above a maximum height; and
does not lower cutting element below a minimum height.

5. The system of claim 1, wherein the cut controller adjusts the height of the cutting element by actuating a hydraulic component which controls the height of the cutting element.

6. The system of claim 1, wherein the preestablished percentage is equal to or less than ten percent of the target value.

7. The system of claim 1, further including a user interface configured to facilitate a user communicating to the cut controller the target value for the property of the plant material.

8. A system comprising:
a swather machine configured to cut a plant material, the swather including a cutting mechanism having a cutting element configured to cut the plant material at a particular cut height;
a near-infrared testing system configured to emit near-infrared radiation and receive a reflected response from the plant material cut at the particular cut height, analyze the reflected response, and generate evaluation data including an actual value for a property of the plant material cut at the particular cut height; and
a cut controller operably coupled to the cutting element to control the particular cut height at which the plant material is cut based at least in part on a comparison of the actual value for the property and a target value for the property, including—
adjusting by the cut controller a height of the cutting element to change the particular cut height if the actual value for the property is substantially different from the target value, and
maintaining by the cut controller the particular cut height if the actual value for the property is within a preestablished percentage of the target value.

9. The system of claim 8, wherein the property of the plant material is selected from the group consisting of: a relative feed value, a protein content value, a fiber content value, a total digestible nutrient value, an acid detergent fiber value, and a neutral detergent fiber value.

10. The system of claim 8, wherein the cut controller adjusts the height of the cutting element by—
raising the cutting element to increase the particular cut height if the actual value for the property is below the target value, and
lowering the cutting element to decrease the particular cut height if the actual value for the property is above the target value.

11. The system of claim 8, wherein the cut controller—
does not raise the cutting element above a maximum height; and
does not lower cutting element below a minimum height.

12. The system of claim 8, wherein the cut controller raises and lower the cutting element by actuating a hydraulic component which controls a height of the cutting element.

13. The system of claim 8, wherein the preestablished percentage is equal to or less than ten percent of the target value.

14. The system of claim 8, further including a user interface configured to facilitate a user communicating to the cut controller the target value for the property of the plant material.

15. A method comprising:
cutting with a swather machine a plant material at a particular cut height;
receiving and analyzing with a near-infrared testing system near-infrared radiation reflected by the plant material cut at the particular cut height, and generating evaluation data including an actual value for a property of the plant material cut at the particular cut height; and
controlling with a cut controller the particular cut height at which the plant material, including comparing the actual value for the property and a target value for the property and—
adjusting by the cut controller a height of the cutting element to change the particular cut height if the actual value for the property is substantially different from the target value, and
maintaining by the cut controller the particular cut height if the actual value for the property is within a preestablished percentage of the target value.

16. The method of claim 15, wherein the property of the plant material is selected from the group consisting of: a relative feed value, a protein content value, a fiber content value, a total digestible nutrient value, an acid detergent fiber value, and a neutral detergent fiber value.

17. The method of claim 15, wherein the cut controller adjusts the height of the cutting element by—
raising the cutting element to increase the particular cut height if the actual value for the property is below the target value, and
lowering the cutting element to decrease the particular cut height if the actual value for the property is above the target value.

18. The method of claim 15, wherein the cut controller—
does not raise the cutting element above a maximum height; and
does not lower cutting element below a minimum height.

19. The method of claim 15, wherein the cutting element is raised and lowered by the cut controller actuating a hydraulic component which controls a height of the cutting element.

20. The method of claim 15, wherein the preestablished percentage is equal to or less than ten percent of the target value.
